(19) United States
(12) Patent Application Publication
(10) Pub. No.: US 2024/0091592 A1
(43) Pub. Date: Mar. 21, 2024

(54) METHOD AND SERVER FOR PROVIDING EXERCISE MANAGEMENT SOLUTION

(71) Applicant: Avant Solution Inc., Cheongju-si (KR)

(72) Inventor: Sang Chul LEE, Yongin-si (KR)

(73) Assignee: Avant Solution Inc., Cheongju-si (KR)

(21) Appl. No.: 18/470,954

(22) Filed: Sep. 20, 2023

(30) Foreign Application Priority Data
Sep. 21, 2022 (KR) 10-2022-0119239

(51) Int. Cl. A63B 24/00

(52) U.S. Cl. CPC A63B 24/0062; A63B 2220/05; A63B 2220/833; A63B 2225/50

(57) ABSTRACT

The present invention relates to a server and a method for providing an exercise management solution to a user. Furthermore, one embodiment of the present invention relates to technology applying the look-up table (LUT). Furthermore, one embodiment of the present invention relates to technology applying artificial intelligence.

### CROSS-REFERENCE TO RELATED APPLICATION

This application claims priority to and the benefit of Korean Patent Application No. 10-2022-0119239, filed on Sep. 21, 2022, the disclosure of which is incorporated herein by reference in its entirety.

### BACKGROUND OF THE DISCLOSURE

### 1. Field of the Disclosure

The present disclosure relates to a server and a method for providing an exercise management solution to a user.

Furthermore, one embodiment of the present disclosure relates to technology applying the look-up table (LUT).

Furthermore, one embodiment of the present disclosure relates to technology applying artificial intelligence.

### 2. Description of the Related Art

The home training population began to increase even before COVID-19 became prevalent, and has increased noticeably since 2020 when COVID-19 became prevalent, and thus take up a large portion of exercise population. As a result, digital healthcare products and applications that assist with the home training are being released.

### 3. Prior Art Literature

### Patent Literatures

(Patent Document 1) Korean Patent Application Publication No. 10-2022-0043364 A (Apr. 5, 2022)
(Patent Document 2) Korean Patent Application Publication No. 10-2022-0061511 A (May 13, 2022)
(Patent Document 3) Korean patent No. 10-2000763 B1 (Jul. 10, 2019)

### SUMMARY OF THE DISCLOSURE

Various embodiments of the present disclosure provide a method and a server for providing an AI based exercise management solution to a user.

The technical purposes sought to be achieved in the present disclosure are not limited to the technical purposes as mentioned above. Other technical purposes not mentioned may be clearly understood by those skilled in the art in the technical field to which the present disclosure belongs from the description below.

According to one embodiment of the present disclosure, a method in which a server provides an AI-based exercise management solution is provided. The method includes receiving, from a user device of a user, hit point timing information, skeleton degree information, and vital sign information of each of a plurality of time periods of the user's exercise video captured by a camera of the user device; performing an exercise training process or an exercise strength management process, based on the hit point timing information, the skeleton degree information, and the vital sign information of each of the plurality of time periods; deriving result information as a result of performing the exercise training process or the exercise strength management process; and transmitting the derived result information to the user device, wherein the exercise video records therein an image of the user performing a specific exercise in which a specific motion set is repeated, for each of the plurality of time periods, wherein the hit point timing information indicates a timing difference between a reference time-point at which a preset major motion of an exercise is performed by the user and an actual time-point at which the user actually performs the preset major motion, wherein the skeleton degree information indicates a difference between a first reference value and an actual measurement value of an angle of a part of a body of the user performing the preset major motion, wherein the vital sign information indicates a difference between a second reference value and an actual measurement value of a vital sign value at a time-point at which the user performs the major motion.

In one embodiment, a first time period among the plurality of time periods represents a time period for which the user performs the specific motion set, wherein a preset look-up table is applied to the first time period of the exercise video to drive the reference time-point, the first reference value, and the second reference value.

In one embodiment, the reference time-point is derived by applying the look-up table preset based on a gender, an age, and a BMI (Body Mass Index) of the user to the first time period of the exercise video, wherein the actual performing time-point (the time-point at which the user actually performs the preset major motion) may be derived based on an acceleration value received in real time from an accelerometer sensor attached to exercise equipment and an angular velocity value received in real time from a gyroscope sensor attached thereto.

In one embodiment, the first reference value may be derived by applying the look-up table preset based on the user's gender, age, and BMI to the first time period of the exercise video, wherein the actual angle measurement value may represent the angle of a part of the user's body performing the major motion in the exercise video.

In one embodiment, the second reference value may be derived by applying the look-up table preset based on the user's gender, age, and BMI to the first time period of the exercise video, wherein the actual vital measurement value may be derived based on the vital value received in real time from a heart rate sensor attached to the user.

In one embodiment, the exercise training process may be determined based on the user's exercise evaluation score, wherein the exercise evaluation score may be determined based on the hit point timing information, the skeleton degree information, and the vital sign information.

In one embodiment, result information obtained by performing the exercise strength management process may be determined based on whether the user has achieved a preset completion condition of the specific exercise and the vital value received in real time from a heart rate sensor attached to the user.

In one embodiment, upon determination that the user fails to achieve the preset completion condition, the user's physical fitness level may be adjusted. The preset look-up table may be adjusted in consideration of the adjusted physical fitness level. An exercise difficulty of a sample image guiding the specific exercise may be adjusted in consideration of the adjusted physical fitness level. Upon determination that the user has achieved the preset completion condition, the vital value of the user may be identified. Whether to recommend a higher-level exercise than the specific exercise may be determined depending on the identified vital value.

In one embodiment, a first time period among the plurality of time periods represents a time period for which the user performs the specific motion set. A preset look-up table may be applied to the first time period of the exercise video to derive the reference performing time-point (the time-point at which the preset major motion of the exercise is performed by the user), the first reference value, and the second reference value. In this regard, the reference performing time-point may be derived by applying the look-up table preset based on the user's gender, age, and BMI to the first time period of the exercise video. The actual performing time-point (the time-point at which the user actually performs the preset major motion) may be derived based on an acceleration value received in real time from an accelerometer sensor attached to exercise equipment and an angular velocity value received in real time from a gyroscope sensor attached thereto. The first reference value may be derived by applying the look-up table preset based on the user's gender, age, and BMI to the first time period of the exercise video. The actual angle measurement value may represent the angle of a part of the user's body performing the major motion in the exercise video. The second reference value may be derived by applying the look-up table preset based on the user's gender, age, and BMI to the first time period of the exercise video. The actual vital measurement value may be derived based on the vital value received in real time from a heart rate sensor attached to the user. The exercise training process may be determined based on the user's exercise evaluation score. The exercise evaluation score may be determined based on the hit point timing information, the skeleton degree information, and the vital sign information. Result information obtained by performing the exercise strength management process may be determined based on whether the user has achieved a preset completion condition of the specific exercise and the vital value received in real time from a heart rate sensor attached to the user. Upon determination that the user fails to achieve the preset completion condition, the user's physical fitness level may be adjusted. The preset look-up table may be adjusted in consideration of the adjusted physical fitness level. An exercise difficulty of a sample image guiding the specific exercise may be adjusted in consideration of the adjusted physical fitness level. Upon determination that the user has achieved the preset completion condition, the vital value of the user may be identified. Whether to recommend a higher-level exercise than the specific exercise may be determined depending on the identified vital value.

According to one embodiment of the present disclosure, a server that provides an AI-based exercise management solution is provided. The server includes a transceiver; and a processor, wherein the processor is configured to: control the transceiver to receive, from a user device of a user, hit point timing information, skeleton degree information, and vital sign information of each of a plurality of time periods of the user's exercise video captured by a camera of the user device; perform an exercise training process or an exercise strength management process, based on the hit point timing information, the skeleton degree information, and the vital sign information of each of the plurality of time periods; derive result information as a result of performing the exercise training process or the exercise strength management process; and control the transceiver to transmit the derived result information to the user device, wherein the exercise video records therein an image of the user performing a specific exercise in which a specific motion set is repeated, for each of the plurality of time periods, wherein the hit point timing information indicates a timing difference between a reference time-point at which a preset major motion of an exercise is performed by the user and an actual time-point at which the user actually performs the preset major motion, wherein the skeleton degree information indicates a difference between a first reference value and an actual measurement value of an angle of a part of a body of the user performing the preset major motion, wherein the vital sign information indicates a difference between a second reference value and an actual measurement value of a vital sign value at a time-point at which the user performs the major motion.

According to various embodiments of the present disclosure, the server may effectively provide the AI-based exercise management solution to the user.

The effects of the present disclosure are not limited to those described above, and other effects not described may be clearly understood by those skilled in the art in the technical field to which the present disclosure belongs from the following descriptions.

### BRIEF DESCRIPTION OF THE DRAWINGS

Other aspects, features and benefits as described above of certain preferred embodiments of the present disclosure will become more apparent from the following descriptions taken in conjunction with the accompanying drawings:

FIG. 1 shows an example of a communication structure between a server and a user device.

FIG. 2 is a flowchart showing an operation of the server according to one embodiment;

FIGS. 3A, 3B and 3C are an example that describes an overall process in which the server and the user device provide an AI-based exercise management solution to a user according to an embodiment;

FIGS. 4A and 4B show an example of an exercise training process and an exercise strength management process provided from the server to the user;

FIGS. 5A and 5B show an example of a whole-body balanced development exercise management process, a physical fitness balanced development management process, an exercise schedule management process, and an exercise encouragement and recommendation process provided from the server to the user;

FIGS. 6A and 6B show an example of a configuration of each of the user device and the server that provides an AI-based exercise management solution to the user;

FIG. 7 is a block diagram showing a configuration of the server according to one embodiment; and

FIG. 8 is a block diagram showing a configuration of a processor according to one embodiment.

### DETAILED DESCRIPTION OF THE DISCLOSURE

The present disclosure may have various changes and have several embodiments, and thus is intended to illustrate specific embodiments in the drawings and describe them in detail. However, this is not intended to limit the present disclosure to a specific embodiment. It should be understood that the present disclosure includes all changes, equivalents, or substitutes included in the idea and technical scope of the present disclosure.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

It will be understood that when an element or layer is referred to as being “connected to”, or “connected to” another element or layer, it may be directly on, connected to, or connected to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “including”, “include”, and “including” when used in this specification, specify the presence of the stated features, integers, operations, elements, and/or components, but do not preclude the presence or addition of one or more other features, integers, operations, elements, components, and/or portions thereof.

Furthermore, terms such as “unit”, and “module” as described herein may mean a unit that processes at least one function or operation, and may be implemented in a hardware or software manner, or in a combination of hardware and software.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, with reference to the attached drawings, preferred embodiments of the present disclosure will be described in more detail. In order to facilitate overall understanding in describing the present disclosure, the same reference signs are used for the same components in the drawings, and duplicate descriptions of the same components are omitted.

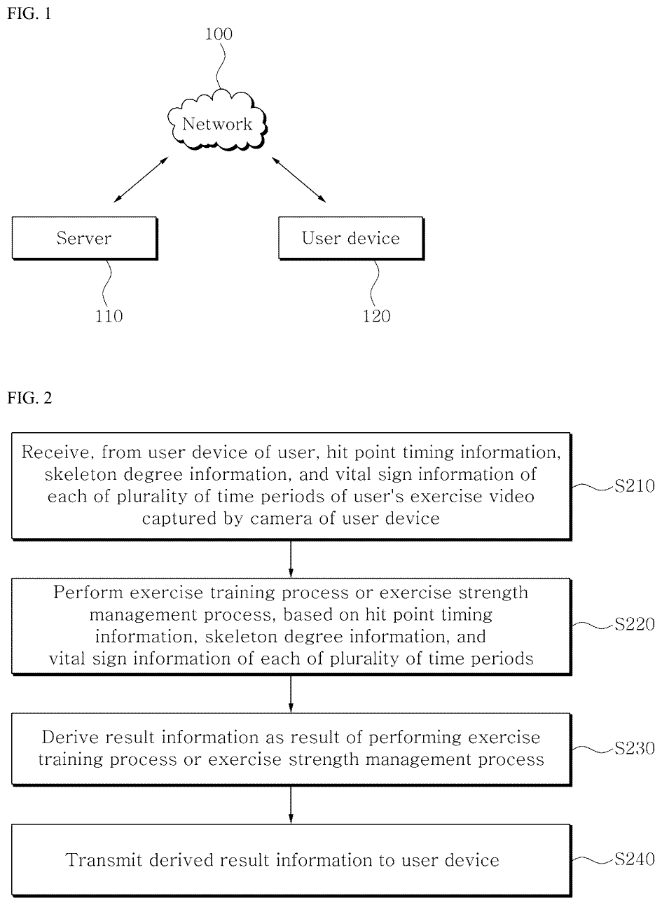

FIG. 1 shows an example of a communication structure between a server and a user device.

As used herein, the term “a server” may be a type of a server that communicates with an external device (e.g., a user device) or an external server (other than the server) via a network and may represent a server for operating the external device and server. In one example, the server may be implemented using a web the server program that is provided on general hardware for a server in various ways depending on an operating system such as DOS, Windows, Linux, Unix, and Macintosh.

In one embodiment, the server 110 may refer to a device that provides an AI-based exercise management solution to a user. For example, the server 110 may be referred to by various names, such as an AI-based exercise management platform, a digital exercise management platform, a digital exercise management platform the server, and an AI-based exercise management the server.

As used herein, the term “a user device” may be implemented as a variety of devices including smartphones, cell phones, smart TVs, smart watches, electronic wristwatches, set-top boxes, tablet PCs, digital cameras, camcorders, laptop computers, desktops, e-readers, digital broadcasting terminals, PDAs (Personal Digital Assistants), PMPs (Portable Multimedia Players), navigation devices, MP3 players, wearable devices, air conditioners, microwave ovens, audio devices, DVD players, etc.

In accordance with the present disclosure, the server 110 and the user device 120 may communicate with each other over a communication network. The communication network may be, for example, a wireless communication network or a wired communication network. The wireless communication network may be, for example, a communication network based on Long Term Evolution (LTE) Radio Access Technology (RAT), New Radio (NR) RAT, WiFi, etc.

In one example, the server 110 and the user device 120 may perform device-to-device (D2D) communication with each other and may perform UU communication with a base station (e.g., gNB and/or eNB). In time division multiple access (TDMA) and frequency division multiple access (FDMA) systems for performing the D2D communication or the UU communication, accurate time and frequency synchronization may be required. When the time and frequency synchronization is not accurate, system performance may deteriorate due to inter-symbol interference (ISI) and inter-carrier interference (ICI). In the D2D communication according to one example, for the time/frequency synchronization, a synchronization signal may be used in a physical layer, and a master information block (MIB) may be used in a radio link control (RLC) layer.

In the D2D communication process, the server 110 and the user device 120 may be synchronized directly with GNSS (global navigation satellite systems), or may be indirectly synchronized with GNSS via a terminal (within network coverage or outside the network coverage) that is directly synchronized with GNSS. When GNSS is set as a synchronization source, the server 110 and the user device 120 may calculate a DFN and a subframe number using Coordinated Universal Time (UTC) and a (pre)set Direct Frame Number (DFN) offset.

Alternatively, the server 110 and the user device 120 may be synchronized directly with the base station or with another terminal which is time/frequency-synchronized with the base station. For example, the base station may be an eNB or gNB. For example, when the server 110 and the user device 120 are within the network coverage, the server 110 and the user device 120 may receive synchronization information provided from the base station, and thus may be synchronized directly with the base station. Afterwards, the server 110 and the user device 120 may provide the synchronization information to other adjacent terminals thereto. When a base station timing is set based on synchronization, the server 110 and the user device 120 may follow a cell associated with a related frequency (when being within the cell coverage at the frequency), a primary cell, or a serving cell (when being outside the cell coverage at the frequency), for synchronization and downlink measurement.

The base station (e.g., serving cell) may provide synchronization setting for a carrier used for the D2D or sidelink (SL) communication. In this case, the server 110 and the user device 120 may follow the synchronization setting received from the base station. When the server 110 and the user device 120 have not detected any cell on the carrier used for the D2D or SL communication and have not receive the synchronization setting from the serving cell, the server 110 and the user device 120 may follow preset synchronization setting.

Alternatively, the server 110 and the user device 120 may be synchronized with another terminal that has not acquired the synchronization information directly or indirectly from a base station or GNSS. The synchronization source and a preference may be preset for the terminal. Alternatively, the synchronization source and the preference may be set based on a control message provided from the base station.

In one embodiment, the server 110 and the user device 120 may communicate with each other over a network 100 (or communication network). For example, when the server 110 transmits data to the network 100, the network 100 may deliver (transmit) the data to the user device 120. Alternatively, when the user device 120 transmits data to the network 100, the network 100 may deliver (transmit) the data to the server 110.

In following descriptions of FIGS. 2 to 6, a method in which the server 110 provides an AI-based exercise management solution to the user based on signal exchange or communication between the server 110 and the user device 120 will be described in detail.

FIG. 2 is a flowchart showing an operation of the server according to one embodiment.

Operations as disclosed in the flowchart of FIG. 2 may be performed in combination with various embodiments of the present disclosure. In one example, a server as described in FIG. 2 may correspond to the server 110 as shown in FIG. 1 or a server 700 as shown in FIG. 7. In one example, the operations as disclosed in the flowchart of FIG. 2 may correspond to some of operations (of the server) as disclosed in FIG. 1 and FIGS. 3 to 8.

In operation S210, the server according to one embodiment may receive from the user device of the user, hit point timing information, skeleton degree information, and vital sign information of each of a plurality of time periods of the user's exercise video captured by a camera of the user device.

In operation S220, the server according to an embodiment may perform an exercise training process or an exercise strength management process based on at least one of the hit point timing information, the skeleton degree information, and the vital sign information of each of the plurality of time periods.

In operation S230, the server according to one embodiment may derive result information as a result of performing the exercise training process or the exercise strength management process.

In operation S240, the server according to one embodiment may transmit the derived result information to the user device.

In one embodiment, the exercise video may represent a video capturing the user performing a specific exercise in which a specific motion set is repeated.

In one example, the specific exercise may be an exercise using a kettle bell (e.g., a kettle bell swing). In another example, the specific exercise may mean a squat or a jumping jack.

In one example, when the specific exercise is a kettle bell swing, the specific motion set may mean a series of motions of moving the kettle bell back and then moving the same forward to reach a hit point as a peak point and then moving the same back. One time period among the plurality of time periods may mean a time period for which the specific motion set is performed. The plurality of time periods may refer to a total time period for which the specific motion set is performed a target number of times (e.g., 15 times).

In one embodiment, the hit point timing information may indicate a timing difference between a reference time-point at which a preset major motion of an exercise is performed by the user and an actual time-point at which the user actually performs the preset major motion.

For example, the preset major motion may refer to a motion in which the user moves the kettle bell forward to reach the peak point in the above example.

In one embodiment, the skeleton degree information may indicate a difference between a first reference value of an angle of a part of a body of the user performing the preset major motion and an actual angle measurement value of an angle of a part of a body of the user performing the preset major motion.

For example, when the specific exercise is a kettle bell swing, the angle of the part of the user's body may mean an angle defined between an arm and a spine of the user or an angle defined between the spine and a thigh of the user.

In one embodiment, the vital sign information may indicate a difference between a second reference value of a vital sign value at a time-point at which the user performs the major motion and an actual vital measurement value at a time-point at which the user performs the major motion.

For example, when the specific exercise is the kettle bell swing, the vital sign information indicates a difference between the second reference value of the vital sign value at the time-point at which the user performs the major motion in which the user moves the kettle bell forward to reach the peak point, and the actual vital measurement value (i.e., a heart rate measurement value) at the time-point at which the user performs the major motion in which the user moves the kettle bell forward to reach the peak point.

In one embodiment, a first time period among the plurality of time periods represents a time period for which the user performs the specific motion set. A preset look-up table may be applied to the first time period of the exercise video to derive the reference performing time-point (the time-point at which the preset major motion of the exercise is performed by the user), the first reference value, and the second reference value.

In one embodiment, the reference performing time-point may be derived by applying the look-up table preset based on the user's gender, age, and BMI to the first time period of the exercise video. The actual performing time-point (the time-point at which the user actually performs the preset major motion) may be derived based on an acceleration value received in real time from an accelerometer sensor attached to exercise equipment and an angular velocity value received in real time from a gyroscope sensor attached thereto.

In one example, the exercise equipment may be a kettle bell.

In one embodiment, the first reference value may be derived by applying the look-up table preset based on the user's gender, age, and BMI to the first time period of the exercise video. The actual angle measurement value may represent the angle of a part of the user's body performing the major motion in the exercise video.

In one embodiment, the second reference value may be derived by applying the look-up table preset based on the user's gender, age, and BMI to the first time period of the exercise video. The actual vital measurement value may be derived based on the vital value received in real time from a heart rate sensor attached to the user.

In one embodiment, the exercise training process may be determined based on the user's exercise evaluation score. The exercise evaluation score may be determined based on the hit point timing information, the skeleton degree information, and the vital sign information.

In one embodiment, result information obtained by performing the exercise strength management process may be determined based on whether the user has achieved a preset completion condition of the specific exercise and the vital value received in real time from a heart rate sensor attached to the user.

In one embodiment, upon determination that the user fails to achieve the preset completion condition, the user's physical fitness level may be adjusted. The preset look-up table may be adjusted in consideration of the adjusted physical fitness level. An exercise difficulty of a sample image guiding the specific exercise may be adjusted in consideration of the adjusted physical fitness level. Upon determination that the user has achieved the preset completion condition, the vital value of the user may be identified. Whether to recommend a higher-level exercise than the specific exercise may be determined depending on the identified vital value.

In one example, the preset completion condition may be achieving 15 kettle bell swings.

In one example, when the user fails to achieve the preset completion condition, the user's physical fitness level may be adjusted downwardly from a high level to a middle level, and the exercise difficulty of the sample image guiding the kettle bell swing may be lowered.

In one example, when the specific exercise is the kettle bell swing, the higher-level exercise may be a barbell exercise.

In one embodiment, a first time period among the plurality of time periods represents a time period for which the user performs the specific motion set. A preset look-up table may be applied to the first time period of the exercise video to derive the reference performing time-point (the time-point at which the preset major motion of the exercise is performed by the user), the first reference value, and the second reference value. In this regard, the reference performing time-point may be derived by applying the look-up table preset based on the user's gender, age, and BMI to the first time period of the exercise video. The actual performing time-point (the time-point at which the user actually performs the preset major motion) may be derived based on an acceleration value received in real time from an accelerometer sensor attached to exercise equipment and an angular velocity value received in real time from a gyroscope sensor attached thereto. The first reference value may be derived by applying the look-up table preset based on the user's gender, age, and BMI to the first time period of the exercise video. The actual angle measurement value may represent the angle of a part of the user's body performing the major motion in the exercise video. The second reference value may be derived by applying the look-up table preset based on the user's gender, age, and BMI to the first time period of the exercise video. The actual vital measurement value may be derived based on the vital value received in real time from a heart rate sensor attached to the user. The exercise training process may be determined based on the user's exercise evaluation score. The exercise evaluation score may be determined based on the hit point timing information, the skeleton degree information, and the vital sign information. Result information obtained by performing the exercise strength management process may be determined based on whether the user has achieved a preset completion condition of the specific exercise and the vital value received in real time from a heart rate sensor attached to the user. Upon determination that the user fails to achieve the preset completion condition, the user's physical fitness level may be adjusted. The preset look-up table may be adjusted in consideration of the adjusted physical fitness level. An exercise difficulty of a sample image guiding the specific exercise may be adjusted in consideration of the adjusted physical fitness level. Upon determination that the user has achieved the preset completion condition, the vital value of the user may be identified. Whether to recommend a higher-level exercise than the specific exercise may be determined depending on the identified vital value.

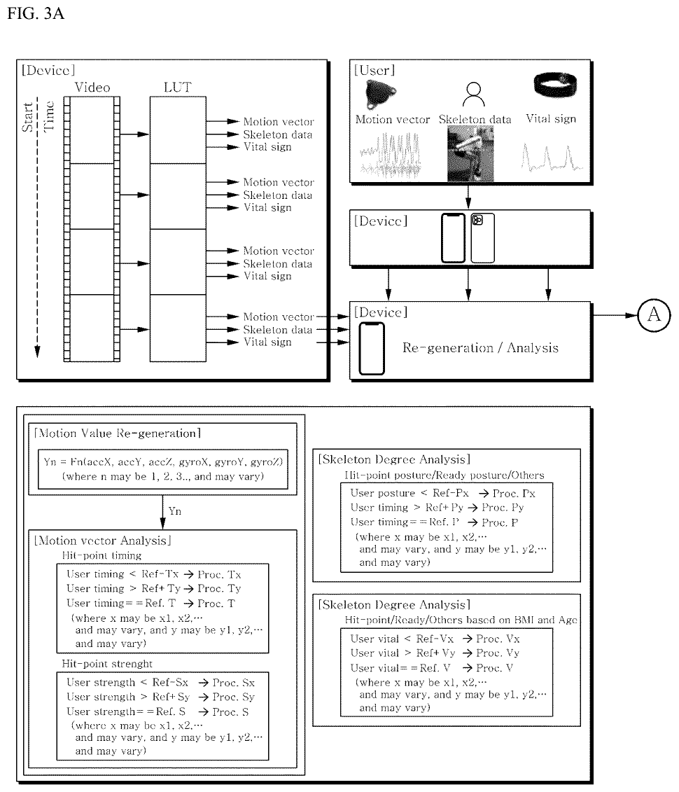

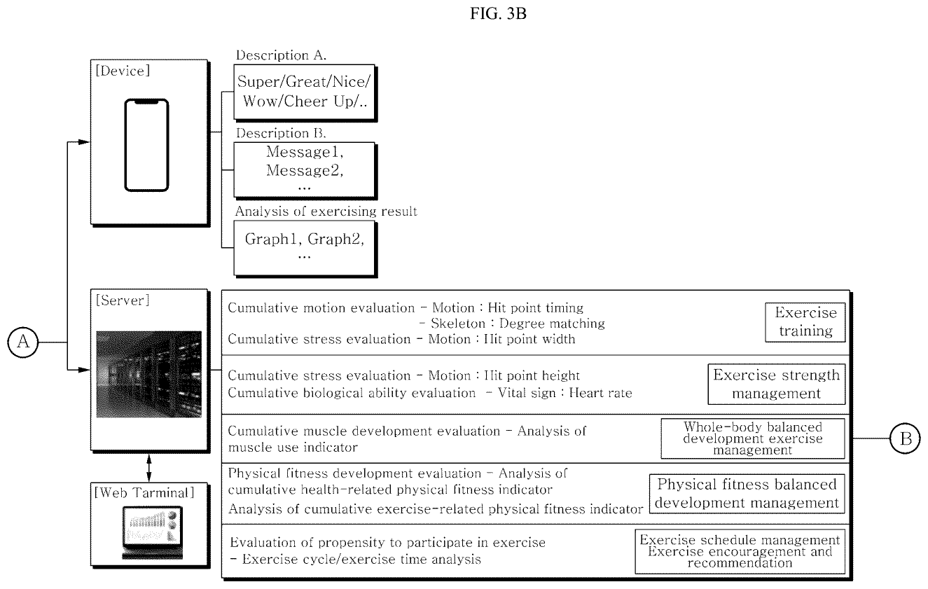

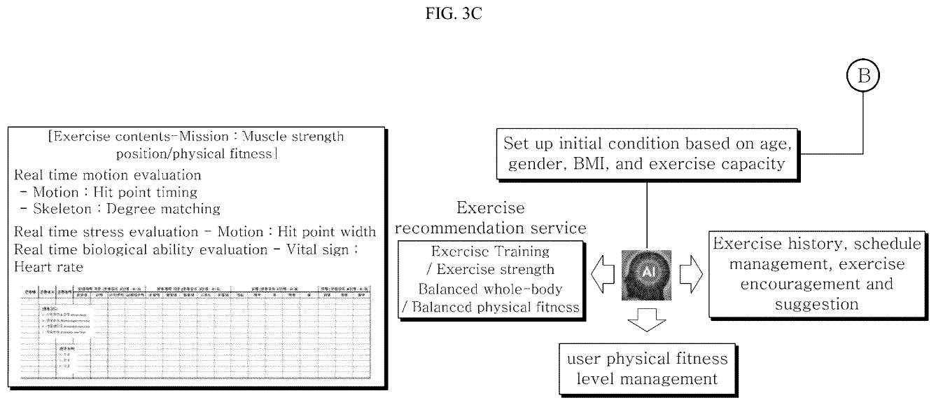

FIGS. 3A, 3B AND 3C are an example that describes an overall process in which the server and the user device provide an AI-based exercise management solution to the user according to an embodiment.

In one embodiment, the user device may image an exercise video that records the user exercising through a camera. The exercise video may be divided into a plurality of time periods, and each time period may mean a time period for which the user performs a specific motion set of a specific exercise. The preset look-up table may be applied to each time period to derive a motion vector, skeleton data, and a vital sign.

In one embodiment, the motion vector, the skeleton data, and the vital sign may be used to derive the reference performing time-point, the first reference value, and the second reference value as described above in FIG. 2, respectively.

In one embodiment, the accelerometer sensor and the gyroscope sensor attached to the exercise device may be used to derive the actual performing time-point as described above in FIG. 2. The heart rate sensor attached to the user may be used to derive the actual vital measurement value as described above in FIG. 2. The user's exercise video may be used to derive the actual angle measurement value as described above in FIG. 2.

In one embodiment, the server may set up an initial condition and set the look-up table based on the user's age, gender, BMI, exercise ability, etc. The server may provide an exercise recommendation service such as exercise training programs, exercise strength whole-body balanced programs, and physical fitness balanced programs, manage exercise history and schedules, encourage the exercise, and manage the user's physical fitness level.

In one embodiment, the user device may derive the hit point timing information, the skeleton degree information, and the vital sign information based on the reference performing time-point, the first reference value, and the second reference value.

In one embodiment, the hit point timing information may indicate a timing difference between a reference time-point at which a preset major motion of an exercise is performed by the user and an actual time-point at which the user actually performs the preset major motion.

In one embodiment, the skeleton degree information may indicate a difference between a first reference value of an angle of a part of a body of the user performing the preset major motion and an actual angle measurement value of an angle of a part of a body of the user performing the preset major motion.

In one embodiment, the vital sign information may indicate a difference between a second reference value of a vital sign value at a time-point at which the user performs the major motion and an actual vital measurement value at a time-point at which the user performs the major motion.

The server according to one embodiment may perform at least one of an exercise training process, an exercise strength management process, a whole-body balanced development exercise management process, a physical fitness balanced development management process, or an exercise schedule management/exercise encouragement and recommendation process, based on at least one of the hit point timing information, the skeleton degree information, and the vital sign information about each of the plurality of time periods.

The user device according to one embodiment may respond to the user's exercise performance (super/great/nice, etc.) and may display a feedback message about the exercise performance, and may display a graph analyzing the exercise result.

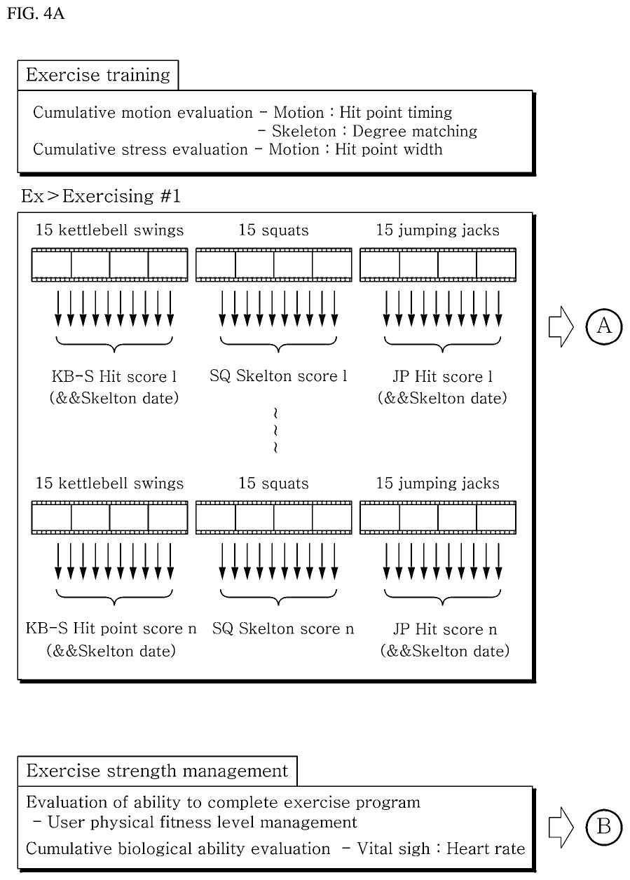

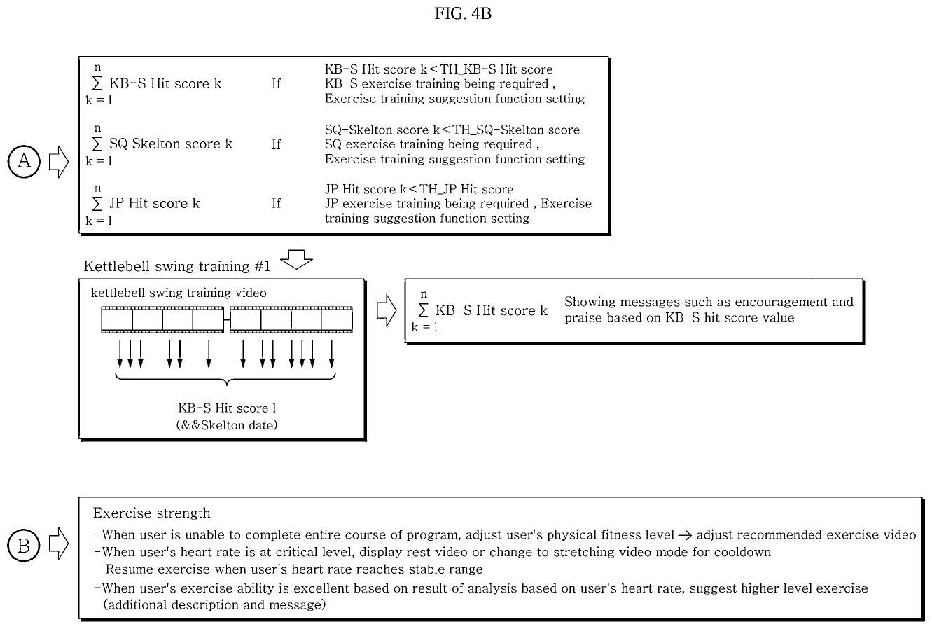

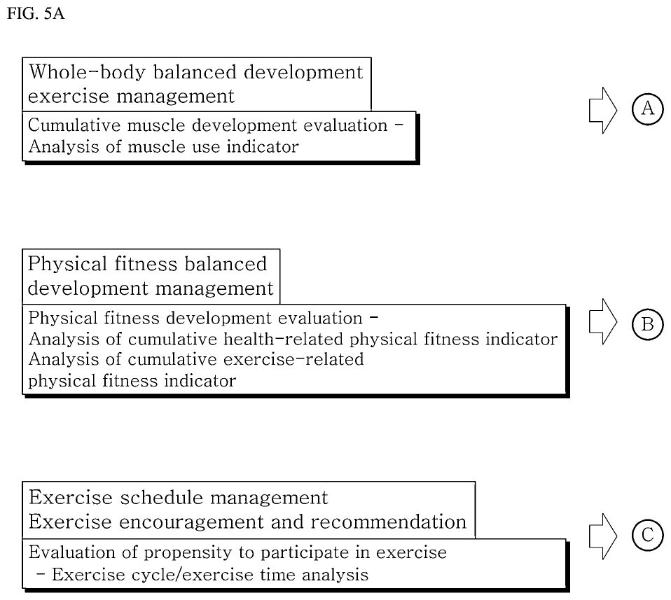

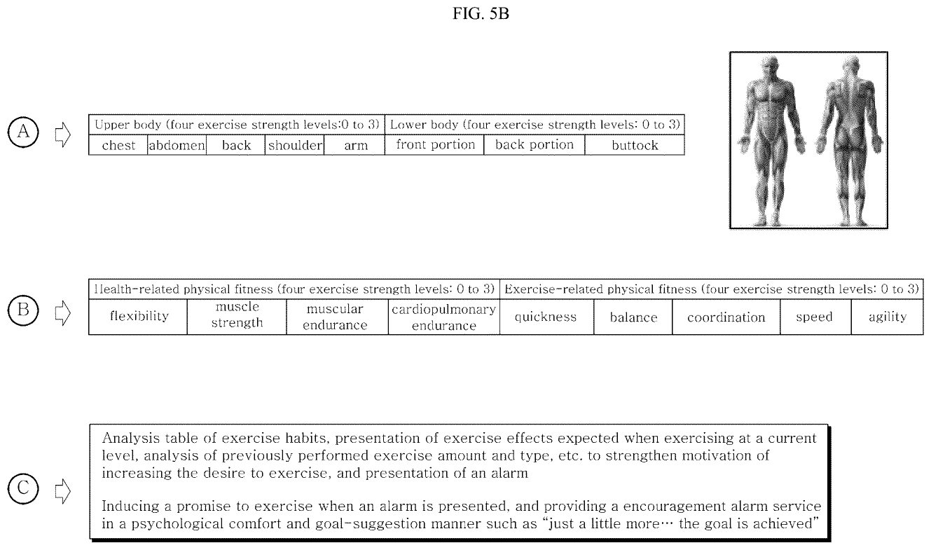

FIGS. 4A and 4B show an example of the exercise training process and the exercise strength management process provided from the server to the user.

The server according to one embodiment may perform an exercise training process based on at least one of the hit point timing information, the skeleton degree information, and the vital sign information of each of the plurality of time periods.

In one embodiment, the server may detect a hit point timing based on the motion vector, and may match the angle based on the skeleton, and perform cumulative motion evaluation based thereon.

In one embodiment, the server may detect a hit point width based on the motion vector, and may perform cumulative strength evaluation based thereon.

In one embodiment, when the specific exercise is a kettle bell swing, each of the plurality of time periods may relate to one kettle bell swing motion. The server may give an evaluation score to each kettle bell swing. When one kettle bell swing among the plurality of kettle bell swings does not exceed a preset threshold evaluation score, the server/user device may inform the user that kettle bell exercise training is necessary, and may set the exercise training recommendation function.

In one embodiment, when the exercise training recommendation function is executed, a kettle bell swing training video image that the user may view and follow may be displayed. An evaluation score may also be given to the exercise motion that the user follows based on the training image. The server/user device may display messages such as encouragement to repeat the training, and praise according to the evaluation score.

The server according to one embodiment may perform an exercise strength management process based on at least one of the hit point timing information, the skeleton degree information, and the vital sign information of each of the plurality of time periods.

In one embodiment, the server may manage the user's physical fitness level based on an evaluating result of the user's ability to complete the exercise program. Cumulative vital capacity assessment may be performed based on the vital sign and the heart rate.

In one embodiment, the exercise strength may be adjusted as follows. When the user is unable to complete an entire course of the program, the user's physical fitness level may be adjusted, and a recommended exercise video may be adjusted. When the user's heart rate is at a critical level, a rest video may be displayed or a stretching video mode for cooldown may be activated. The exercise may be resumed when the user's heart rate reaches a stable range. When the user's exercise ability is excellent based on a result of analysis based on the user's heart rate, a higher-level exercise may be suggested. In this regard, additional description and message may be displayed.

FIGS. 5A and 5B show an example of a whole-body balanced development exercise management process, a physical fitness balanced development management process, an exercise schedule management process, and an exercise encouragement and recommendation process provided from the server to the user.

The server according to one embodiment may perform the whole-body balanced development exercise management process based on at least one of the hit point timing information, the skeleton degree information, and the vital sign information of each of the plurality of time periods. A cumulative muscle development evaluation may be performed based on an analyzing result of a muscle use indicator. Four exercise strength levels (0 to 3) related to an upper body including a chest, an abdomen, a back, a shoulder, and an arm may be analyzed. Four exercise strength levels (0 to 3) related to a lower body including a front portion, a back portion, and a buttock may be analyzed.

The server according to one embodiment may perform the physical fitness balanced development management process based on at least one of the hit point timing information, the skeleton degree information, and the vital sign information of each of the plurality of time periods. The physical fitness development evaluation may be performed based on an analysis result of a cumulative health-related physical fitness indicator and an analysis result of a cumulative exercise-related physical fitness indicator. Four exercise strength levels (0 to 3) of a health-related physical fitness including flexibility, muscle strength, muscular endurance, cardiopulmonary endurance, etc. may be analyzed. Four exercise strength levels (0 to 3) of an exercise-related physical fitness including quickness, balance, coordination, speed, and agility may be analyzed.

The server according to one embodiment may perform the exercise schedule management/exercise encouragement and recommendation process based on at least one of the hit point timing information, the skeleton degree information, and the vital sign information of each of the plurality of time periods. A propensity for participating in the exercise may be evaluated based on an exercise cycle/exercise time analysis result.

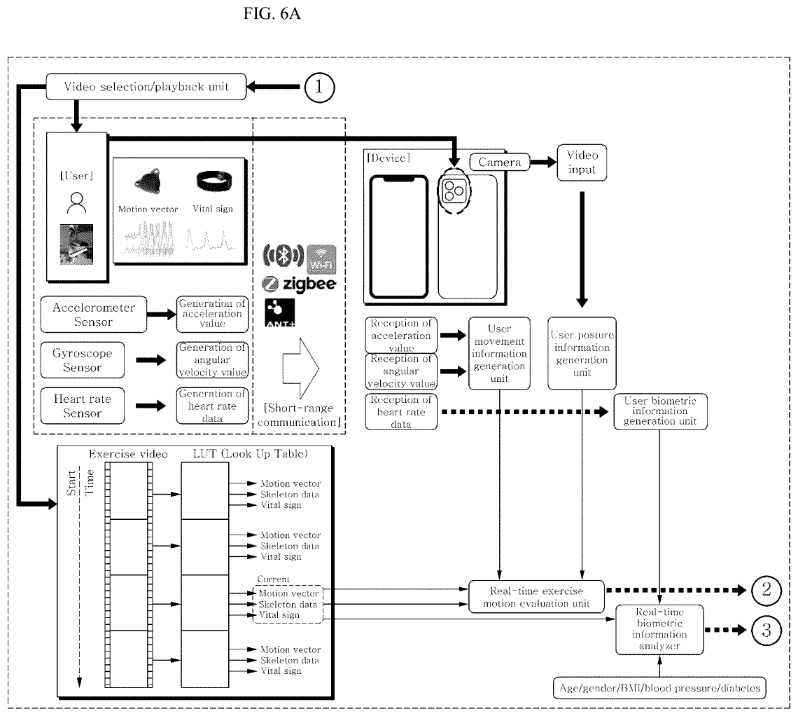

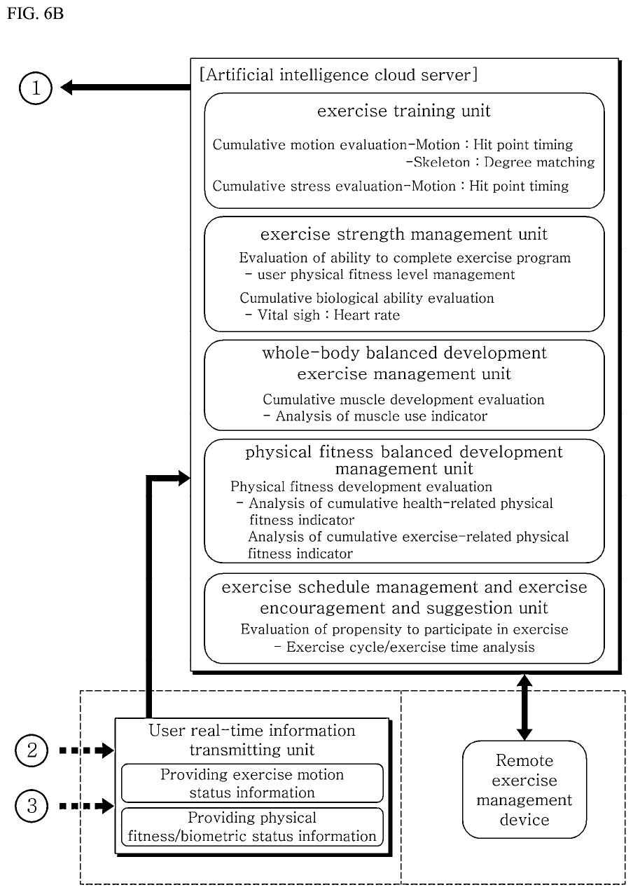

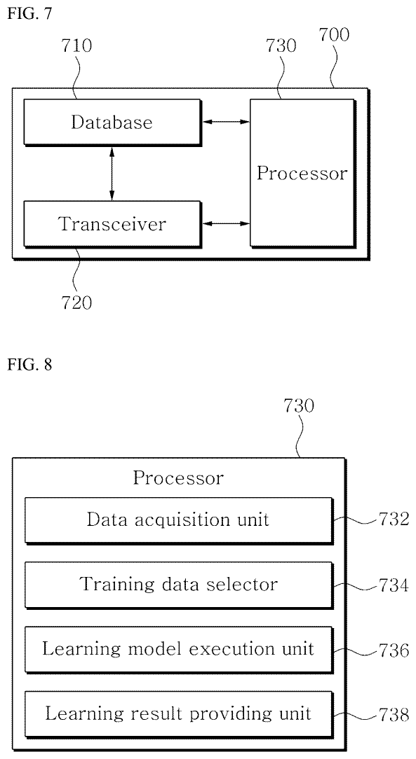

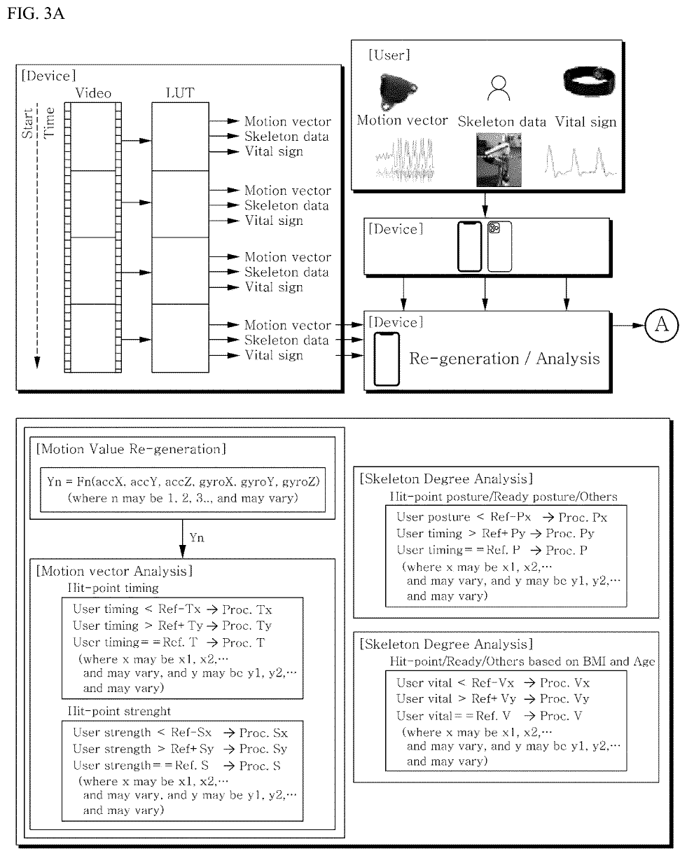

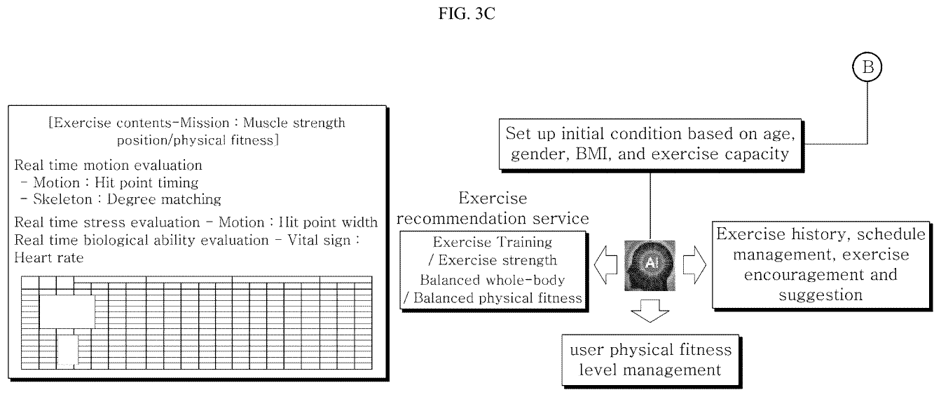

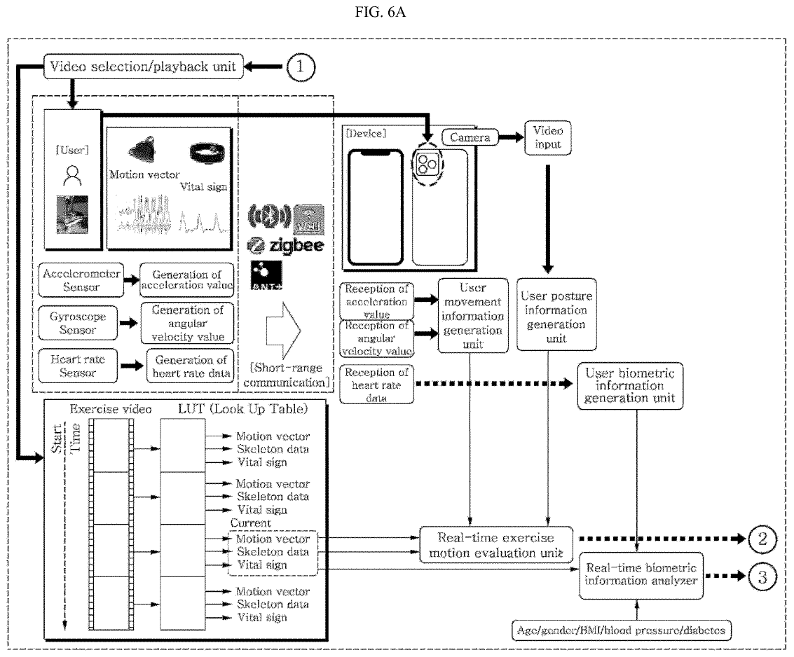

FIGS. 6A and 6B show an example of a configuration of a user device and a server that provides an AI-based exercise management solution to the user.

In one embodiment, a video selection/playback unit of the user device may select and play the user's exercise video, and may acquire the motion vector, the skeleton data, and the vital sign by mapping the exercise video and the look-up table with each other.

In one embodiment, an accelerometer sensor and a gyroscope sensor attached to the exercise device (e.g., a kettle bell), and a heart rate sensor attached to the user may generate an acceleration value, an angular velocity value, and heart rate data, respectively. The data acquired from the sensors may be transmitted to the user device via near-distance communication such as Bluetooth, Zigbee, or Wi-Fi.

In one embodiment, a user movement information generation unit of the user device may generate user movement information based on the received acceleration value and angular velocity value, and may transmit the generated information to a real time exercise motion evaluation unit.

In one embodiment, a user posture information generation unit of the user device may generate user posture information based on the user exercise video acquired through the camera, and may transmit the generated information to the real time exercise motion evaluation unit.

In one embodiment, a user biometric information generation unit of the user device may generate user biometric information based on the received heart rate data, and may transmit the generated information to a real time biometric information analyzer.

In one embodiment, the real-time exercise motion evaluation unit may evaluate the real-time exercise motion based on the received user movement information and user posture information, and the look-up table information of each time period. The real-time exercise motion evaluation unit may transmit the evaluated data to a user real-time information transmitting unit.

In one embodiment, the real-time biometric information analyzer may analyze the user biometric information in real time based on the data such as the age/gender/BMI/blood pressure/diabetes and the look-up table information of each time period. The real-time biometric information analyzer may transmit the analyzed data to the user real-time information transmitting unit.

In one embodiment, the user real-time information transmitting unit of the user device may transmit exercise motion status information and physical fitness/biometric status information to an artificial intelligence cloud server.

In one embodiment, the artificial intelligence cloud server may perform the exercise management process as described above in FIGS. 2 to 5 via an exercise training unit, an exercise strength management unit, a whole-body balanced development exercise management unit, a physical fitness balanced development management unit, and an exercise schedule management and exercise encouragement and suggestion unit. In this regard, the artificial intelligence cloud server may communicate the data in real time with a remote exercise management device.

FIG. 7 is a block diagram showing a configuration of the server according to one embodiment.

As shown in FIG. 7, a server 700 according to one embodiment may include database (DB) 710, a transceiver 720, and a processor 730. However, in some cases, not all of the components as shown in FIGS. 5A and 5B may be essential components of the server 700. The server 700 may be composed of a larger or smaller number of components than those as shown in FIGS. 5A and 5B.

In the server 700 according to one embodiment, the database 710, the transceiver 720, and the processor 730 may be respectively implemented as separate chips, or at least two or more of the database 710, the transceiver 720, and the processor 730 may be implemented in one chip.

The database (DB) 710 may store therein the data received from the transceiver 720 or the processor 730. In one embodiment, the database 710 may store therein at least one program. In one example, the at least one program may be used when the processor 730 executes a learning model.

The transceiver 720 may be used for communication of data/signal between modules within the server and/or, and may perform communication with an external device. In one example, the transceiver 720 may transmit and receive data to and from the external device (e.g., an external user device, an external server, etc.).

The processor 730 according to one embodiment may control the overall operations of the server 700. In one example, the processor 730 may generally control the database 710 and the transceiver 720 by executing the programs stored in the database 710 of the server 700. In one example, the processor 730 may perform some of the operations of the server 700 in FIGS. 1 to 6 by executing the programs stored in the database of the server 700.

The processor 730 according to an embodiment may control the transceiver 720 to receive, from the user device, the hit point timing information, the skeleton degree information and the vital sign information of each of the plurality of time periods of the user's exercise video captured by the camera of the user device.

The processor 730 according to an embodiment may perform an exercise training process or an exercise strength management process based on at least one of the hit point timing information, the skeleton degree information, and the vital sign information of each of the plurality of time periods.

The processor 730 according to one embodiment may derive result information as a result of performing the exercise training process or the exercise strength management process.

The processor 730 according to one embodiment may control the transceiver 720 to transmit the derived result information to the user device.

In one embodiment, the exercise video records therein an image of the user performing a specific exercise in which a specific motion set is repeated, for each of the plurality of time periods.

In one embodiment, the hit point timing information indicates a timing difference between a reference time-point at which a preset major motion of an exercise is performed by the user and an actual time-point at which the user actually performs the preset major motion.

In one embodiment, the skeleton degree information indicates a difference between a first reference value and an actual measurement value of an angle of a part of a body of the user performing the preset major motion.

In one embodiment, the vital sign information indicates a difference between a second reference value and an actual measurement value of a vital sign value at a time-point at which the user performs the major motion.

In one example, the processor 730 may execute an artificial intelligence (AI) learning model based on at least one program stored in the database 710. Hereinafter, with reference to FIG. 8, an example of a configuration of the processor 730 for executing the AI learning model is described.

FIG. 8 is a block diagram showing a configuration of a processor according to one embodiment.

As shown in FIG. 8, the processor 730 according to one embodiment may include a data acquisition unit 732, a training data selector 734, a learning model execution unit 736, and a learning result providing unit 738. However, in some cases, not all of the components as shown in FIG. 8 may be essential components of the processor 730. The processor 730 may be implemented by a larger or smaller number of components than the components as shown in FIG. 8.

Those skilled in the art will appreciate that the configuration of the processor 730 as shown in FIG. 8 is just an example of modules of the processor 730 that may be used when the server 700 performs machine learning based on an AI learning model, and the server 700 does not necessarily perform the machine learning, and thus, some or all of the modules of the processor 730 shown in FIG. 8 may not be included in the processor 730.

The data acquisition unit 732 according to one embodiment may determine user's intention, and may provide associated information, and may acquire data necessary to recommend an alternative motion. Alternatively, the data acquisition unit 732 may acquire data necessary for training to determine the user's intention, and provide the associated information, and recommend the alternative motion.

In one example, the data acquisition unit 732 may acquire at least one of, for example, user voice, image information, and predetermined context information. In one example, the data acquisition unit 732 may convert the acquired data into a pre-defined format.

The training data selector 734 according to one embodiment may select data necessary for training from the data acquired by the data acquisition unit 732. For example, the training data selector 734 may select the data necessary for training among the data acquired by the data acquisition unit 732, according to predefined criteria for determining the user's intention, providing the associated information, and recommending the alternative motion. Alternatively, the training data selector 734 may select data according to a predefined criterion based on the learning result by the learning model execution unit 736.

The learning model execution unit 736 according to one embodiment may learn the criteria used for determining the user's intention, providing the associated information, and recommending the alternative motion. Furthermore, the learning model execution unit 736 may learn criteria used to determine learned data which are used to determine the user's intention, provide the associated information, and recommend the alternative motion.

Furthermore, the learning model execution unit 736 may train the AI learning model used for determining the user intention, providing the associated information, and recommending the alternative motion using the training data. In this case, the AI learning model may be a pre-constructed model. For example, the AI learning model may be a model that is pre-constructed based on a received basic training data (e.g., sample data, etc.).

The AI learning model may be constructed based on the application field of the learning model, the purpose of learning, or the computer performance of the device. The AI learning model may be, for example, a model based on a neural network. For example, a model such as Deep Neural Network (DNN), Recurrent Neural Network (RNN), and Bidirectional Recurrent Deep Neural Network (BRDNN) may be used as the AI learning model. However, embodiments are not limited thereto.

When there are a plurality of pre-constructed AI learning models, the learning model execution unit 736 according to one embodiment may determine an AI learning model having a high correlation between input training data and the basic training data as the AI learning model to be trained. In this case, the basic training data may be pre-classified based on data type, and the AI learning model may be pre-constructed based on the data type. For example, the basic training data may be pre-classified based on various criteria such as a region where the training data is created, a time the training data is created, a size of the training data, the genre of the training data, a creator of the training data, a type of an object in the training data, etc.

The learning model execution unit 736 according to one embodiment may train the AI learning model using, for example, the learning algorithm including error back-propagation or gradient descent.

The learning model execution unit 736 according to one embodiment may train the AI learning model, for example, via supervised learning using the training data as an input value. Alternatively, the learning model execution unit 736 may train the AI learning model, for example, via unsupervised learning in which the model may learn, by itself and without any other guidance, a type of data needed to determine the user's intention, provide the associated information, and recommend the alternative motion, and thus may discover the criteria used for determining the user's intention, providing the associated information, and recommending the alternative motion. Alternatively, the learning model execution unit 736 may train the AI learning model, for example, via reinforcement learning using feedbacks about whether the results of determining the user's intention, and providing the associated information, and the recommendation result of the alternative motion are correct.

Furthermore, when the AI learning model has been trained, the learning model execution unit 736 may store the trained AI learning model. In this case, the learning model execution unit 736 may store the trained AI learning model in the database 710 of the server 700. Alternatively, the learning model execution unit 836 may store the trained AI learning model in a memory or database of a server or a device connected to the server 700 through a wired or wireless network.

The learning result providing unit 738 according to one embodiment may derive or acquire (machine) learning results based on the AI learning model trained by the learning model execution unit 736. The learning result providing unit 738 may provide the (machine) learning results to the database 710 or the transceiver 720.

The learning result providing unit 738 according to one embodiment may derive the user exercise evaluation score to be used to provide the exercise training program to the user, based on the AI learning model. For example, the AI learning model may derive the exercise evaluation score using the hit point timing information, the skeleton degree information, and the vital sign information as described above in FIG. 2 and FIGS. 3A, 3B AND 3C as input values.

The learning result providing unit 738 according to one embodiment may adjust the user's physical fitness level. For example, when the user is unable to complete the exercise program provided from the server 700, the server 700 may lower the user's physical fitness level in consideration of the user's age, gender, BMI, etc.

Some embodiments may also be implemented in a form of a recording medium containing therein instructions executable by a computer, such as program modules executed by a computer. The computer-readable medium may be any available media that may be accessed by a computer, including both volatile and nonvolatile media, removable and non-removable media. Furthermore, the computer-readable medium may include both a computer storage medium and a communication medium. The computer storage medium may includes both volatile and non-volatile, removable and non-removable media implemented in any method or technology for storage of information such as computer readable instructions, data structures, program modules or other data. The communication medium may typically include computer-readable instructions, data structures, program modules, or other data in a modulated data signal, such as a carrier wave, or other transmission mechanism, and includes any information transmission medium.

Although the preferred embodiments of the present disclosure have been disclosed for illustrative purposes, those skilled in the art will appreciate that various modifications, additions and substitutions are possible, without departing from the scope and spirit of the disclosure as disclosed in the accompanying claims.

What is claimed is:

1. A server for providing an exercise management solution, the server comprising:
a transceiver; and a processor,
wherein the processor is configured to:
control the transceiver to receive, from a user device of a user, hit point timing information, skeleton degree information, and vital sign information of each of a plurality of time periods of the user's exercise video captured by a camera of the user device;
perform an exercise training process or an exercise strength management process, based on the hit point timing information, the skeleton degree information, and the vital sign information of each of the plurality of time periods;
derive result information as a result of performing the exercise training process or the exercise strength management process; and
control the transceiver to transmit the derived result information to the user device,
wherein the exercise video records therein an image of the user performing a specific exercise in which a specific motion set is repeated, for each of the plurality of time periods,
wherein the hit point timing information indicates a timing difference between a reference time-point at which a preset major motion of an exercise is performed by the user and an actual time-point at which the user actually performs the preset major motion,
wherein the skeleton degree information indicates a difference between a first reference value and an actual measurement value of an angle of a part of a body of the user performing the preset major motion,
wherein the vital sign information indicates a difference between a second reference value and an actual measurement value of a vital sign value at a time-point at which the user performs the major motion,
wherein a first time period among the plurality of time periods represents a time period for which the user performs the specific motion set,
wherein a preset look-up table is applied to the first time period of the exercise video to drive the reference time-point, the first reference value, and the second reference value,
wherein the reference time-point is derived by applying the look-up table preset based on a gender, an age, and a BMI (Body Mass Index) of the user to the first time period of the exercise video,
wherein each of the first reference value and the second reference value is derived by applying the look-up table preset based on the gender, the age, and the BMI of the user to the first time period of the exercise video,
wherein the actual time-point is derived based on an acceleration value received in real time from an accelerometer sensor attached to exercise equipment and an angular velocity value received in real time from a gyroscope sensor attached to the exercise equipment,
wherein the actual measurement value of the angle indicates the angle of the part of the body of the user performing the major motion in the exercise video,
wherein the actual measurement value of the vital signal is derived based on a vital value received in real time from a heart rate sensor attached to the user,
wherein the look-up table is mapped to each video imaged for each of the plurality of time periods included in the exercise video to obtain a motion vector, skeleton data, and a vital sign for each of the plurality of time periods.
